(19) United States
(12) Patent Application Publication
(10) Pub. No.: US 2024/0358307 A1
(43) Pub. Date: Oct. 31, 2024

(54) COATING FOR ELECTRODES USED IN ELECTRODERMAL ACTIVITY SENSORS

(71) Applicant: Toyota Motor Engineering & Manufacturing North America, Inc., Plano, TX (US)

(72) Inventors: Paul Donald Schmalenberg, Ann Arbor, MI (US); Frederico Marcolino Quintao Severgnini, Ann Arbor, MI (US); Hossein Hamidi Shishavan, New Britain, CT (US); Ercan Mehmet Dede, Ann Arbor, MI (US); Parveen Singh, Dearborn, MI (US)

(21) Appl. No.: 18/309,333

(22) Filed: Apr. 28, 2023

(51) Int. Cl. A61B 5/263

(52) U.S. Cl. CPC A61B 5/263; A61B 2562/125

(57) ABSTRACT

An electrode assembly structured for use in detecting electrodermal activity (EDA) includes a substrate, at least two separate electrodes affixed to the substrate, and a separate electrically-conductive coating covering each electrode. Each coating includes a base portion and a plurality of unit cells extending from the base portion. The unit cells combine to form a microstructure on an outer surface of the coating. Dimensions and spatial arrangements of the unit cells can be controlled to provide controlled variations along a contour defined by the microstructures, so that the contour conforms to ridges in the skin surfaces of multiple users. This increases electrical conductivity between the skin surfaces and the conductive coating, thereby enhancing electrical contact between the user and the electrodes covered by the coating.

### TECHNICAL FIELD

The subject matter described herein relates to electrodermal activity (EDA) sensors and, more particularly, to an electrode assembly usable in detecting electrodermal activity in an EDA sensor.

### BACKGROUND

It is known to use electrodermal activity (EDA) sensors to acquire data that may be interpreted to indicate or infer the existence any of a variety of physiological and/or psychological conditions in a user. An EDA sensor may use a pair of electrodes (including one electrode for the supply voltage and another electrode serving as a ground) physically separated by an electrical insulator and in physical contact with the user's skin to transmit an electrical current between the electrodes. Based on an estimate of the resistance or conductance encountered during current transmission, an EDA signal may be generated for analysis of the user's state.

Because the spacing between ridges of the skin may vary between users and may even vary along different portions of the skin surface of a single user, it may be difficult to establish close, conformal contact between the user's skin and an electrode without the use of hydrogel or some other wetting mechanism to increase the contact area. In devices such as wearable fitness trackers, the electrodes will saturate over time from the usual human sweat process which serves the same purpose as the wetting gel. This may work reasonably well for wearable items that will not shift from the same patch of skin over long lengths of time. However, wetting-enhanced electrical contact may not be practical for collection of EDA data in cases where skin contact may be brief or where the specific location of EDA measurement may change.

### SUMMARY

In one aspect of the embodiments described herein, an electrode assembly structured for use in detecting electrodermal activity (EDA) is provided. The electrode assembly includes a substrate, at least two separate electrodes affixed to the substrate, and a separate electrically-conductive coating covering each electrode. Each coating includes a base portion and a plurality of unit cells extending from the base portion.

In another aspect of the embodiments described herein, an electrode assembly structured for use in detecting electrodermal activity (EDA) is provided. The electrode assembly includes a substrate, at least two separate electrodes affixed to the substrate, and a separate electrically conductive coating covering each electrode. Each coating includes a base portion and a plurality of unit cells extending from the base portion. In addition, all of the unit cells are formed from a same material. The material forming the unit cells has a first hardness, and each unit cell is spaced apart at least a predetermined distance from any adjacent unit cell so as to provide a gap therebetween. All gaps between adjacent unit cells are filled in with a conductive material having a second hardness less than the first hardness.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

It will be appreciated that for simplicity and clarity of illustration, where appropriate, reference numerals may have been repeated among the different figures to indicate corresponding or analogous elements. Also, similar reference numerals appearing in different views may refer to similar elements appearing in those views. In addition, the discussion outlines numerous specific details to provide a thorough understanding of the embodiments described herein. Those of skill in the art, however, will understand that the embodiments described herein may be practiced using various combinations of these elements.

FIG. 1A is a schematic plan view of an electrode assembly in accordance with an embodiment described herein.

FIG. 1B is a partial cutaway view of the electrode assembly shown in FIG. 1A.

FIG. 2A is a schematic perspective view of an example unit cell in accordance with an embodiment described herein.

FIG. 2B is a schematic plan view of the unit cell shown in FIG. 2A.

FIGS. 3A-3C, in combination, illustrate a method of generating a geometry of a unit cell in accordance with an embodiment described herein

FIG. 4A is a schematic plan view of a portion of an exemplary overall microstructure showing a possible size progression of the constituent unit cells.

FIG. 4B is a schematic side cross-sectional view of a portion of the unit cells shown in FIG. 4A.

FIG. 5 is a schematic front view of a plurality of unit cells of an overall microstructure in accordance with an embodiment described herein, showing progressions in widths of the unit cell contact edges.

FIG. 6 is a schematic cross-sectional side view of an overall microstructure in accordance with an alternative embodiment described herein.

### DETAILED DESCRIPTION

An electrode assembly structured for use in detecting electrodermal activity (EDA) includes a substrate, at least two separate electrodes affixed to the substrate, and a separate electrically conductive coating covering each electrode. Each coating includes a base portion and a plurality of unit cells extending from the base portion. The unit cells combine to form a microstructure on an outer surface of the coating. Dimensions and spatial arrangements of the unit cells can be controlled to provide controlled variations to a contour defined by the microstructure, so that the overall contour may conform to ridges in the skin surfaces of multiple users. This increases electrical conductivity between the skin surfaces and the conductive coating, thereby enhancing electrical contact between the user and the electrodes covered by the coating.

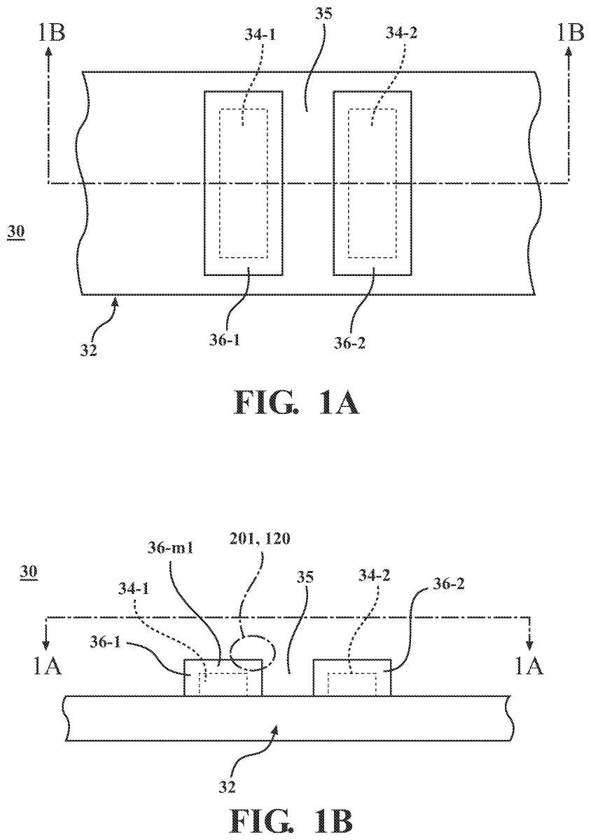

FIGS. 1A-1B show an example of an electrode assembly structured for use in detecting electrodermal activity (EDA) of a user whose skin is in contact with electrodes of the electrode assembly. FIG. 1A is a schematic plan view of an electrode assembly 30 in accordance with an embodiment described herein. FIG. 1B is a partial cutaway view of the electrode assembly 30 shown in FIG. 1A.

In one or more arrangements, the electrode assembly 30 may include a substrate 32. The substrate 32 can be a rigid printed circuit board, flex circuit, or any other host material suitable for attachment of a metallic electrode thereto and having a relatively high electrical resistance.

As used herein, the term “separate electrode” refers to an electrode physically separated from any other electrode by at least one electrically-insulating material interposed between the electrodes. The insulating material may be any suitable material (e.g., an air gap, a non-conductive polymer or other non-conductive material, etc.). For example, FIGS. 1A and 1B show a pair of electrodes 34-1 and 34-2 physically separated by electrically-insulating materials (i.e., an air gap 35 and a portion of substrate 32). Also, as used herein, the term “separate electrically conductive coating” refers to a mass of electrically conductive coating material physically separated from any other mass of electrically conductive coating material by at least one electrically-insulating material interposed between the masses of coating material. The insulating material may be any suitable material (e.g., an air gap, a non-conductive polymer or other non-conductive material, etc.). For example, FIGS. 1A and 1B show a coating 36-1 covering electrode 34-1 and another coating covering electrode 34-2. Each of coatings 36-1 and 36-2 are physically separated from the other one of coatings 36-1 and 36-2 by one or more electrically-insulating materials (i.e., an air gap 35 and a portion of substrate 32).

The electrode assembly 30 may include at least two separate electrodes 34 affixed to the substrate using any suitable method. FIGS. 1A and 1B show an embodiment with a pair of electrodes 34-1, 34-2 affixed to the substrate 32. The electrode(s) 34 may have any structure and composition suitable for use detecting electrodermal activity. For example, the assembly embodiment shown in the drawings includes a pair of parallel rectangular electrodes. However, alternative electrode structures (such as concentric electrodes) may also be used. The electrode(s) 34 may be formed from any suitable metal or metal alloy (e.g., gold, silver, gold alloys, or silver alloys).

A separate electrically conductive coating 36 may be applied to cover each of electrode(s) 34. In one or more arrangements, the coating may 36 be applied so as to completely cover portions of one or more electrodes 34 that are not in physical contact with the substrate 32. Referring to FIGS. 1A-1B, coatings 36-1 and 36-2 may be applied so as to cover respective individual electrodes 34-1 and 34-2 with spaces provided between the associated masses of coating material.

The coatings 36 may be formed from any material which has a relatively low electrical resistance (relative to the electrical currents used in EDA measurements) and which enables resilient deformation or bending responsive to pressure from a finger or palm physically contacting the coating. Examples of suitable coating materials include carbon impregnated silicone rubber and other rubbers (such as fluorosilicone or ethylene propylene diene monomer) containing electrically conductive fillers. Suitable fillers (other than carbon) include silver, aluminum, silver glass, nickel, graphite, and other materials having a relatively high electrical conductivity.

Any embodiment of the coating 36 may include an associated base portion extending between the electrode(s) and substrate along one side thereof, and an overall microstructure (described in greater detail below) supported by the base portion along another side thereof. For example, FIG. 1B shows an embodiment 36m-1 of a base portion of the coating 36-1. The base portion 36m-1 may effectively define an outer surface of the coating 36 that is structured to support an embodiment of an overall microstructure as described herein. In particular arrangements, the coating base portion 36m-1 has a uniform thickness (within applicable tolerances of a molding or other process used to apply the coating).

As used herein, the term “microstructure” refers to a material outer surface structure that can be revealed under magnification higher than 25X. The microstructures described herein may be specialized structures designed to enhance electrical contact between an electrode covered by the coating and a user's skin surface in physical contact with the microstructure. In one or more arrangements, the microstructures described herein may be formed from polymeric materials incorporating conductive filler materials to enhance electrical conductivity. Thus, these microstructures may also be referred to as “conductive microstructures” and/or “polymeric microstructures”.

In one or more arrangements, elements of the microstructures described herein may be formed integrally with an associated base portion, for example, by using a suitable micro-molding process. Alternatively, the microstructures may be applied to the base portion after application of the base portion to the electrode/substrate using a micro-molding process, a controlled deposition process or an additive manufacturing process.

To help provide a strong electrical connection with the stratum corneum of a user's skin, an overall microstructure may be provided along an outer surface of the coating structured to be contacted by a user. Features of this overall microstructure are designed to fill in irregularities in the surface of a finger pad or palm and reduce contact resistance. The overall microstructure formed along the outer surface of the coating may include a plurality of unit cells extending from the base portion. In arrangements described herein, each individual unit cell may form a localized individual microstructure of the coating. In combination, the plurality of unit cells extending from the base portion may form the larger, overall microstructure supported by the base portion. Unit cells of the overall microstructure may be structured and spatially arranged along the coating base portion in any of a variety of ways as described herein, to accommodate and conform to the variable widths of (and spacings between) ridges of a user's skin surface.

A plurality of unit cells 120 may be incorporated into an outer surface of a coating applied to an electrode as described herein. Each unit cell may be structured to extend between ridges and irregularities in the skin surface of a finger pad or palm of a user touching the microstructure, and to intimately contact the skin surface between these ridges and irregularities. This may aid in reducing contact resistance between the skin surface and the electrode covered by the coating. In the following description, the unit cells 120 will be described with reference to FIG. 1B, as forming a part of an overall microstructure 201 extending along the outer surface of coating 36-1. However, it will be understood that the microstructures described herein may be applied to any embodiment of a coating outer surface structured to contact the skin of a user.

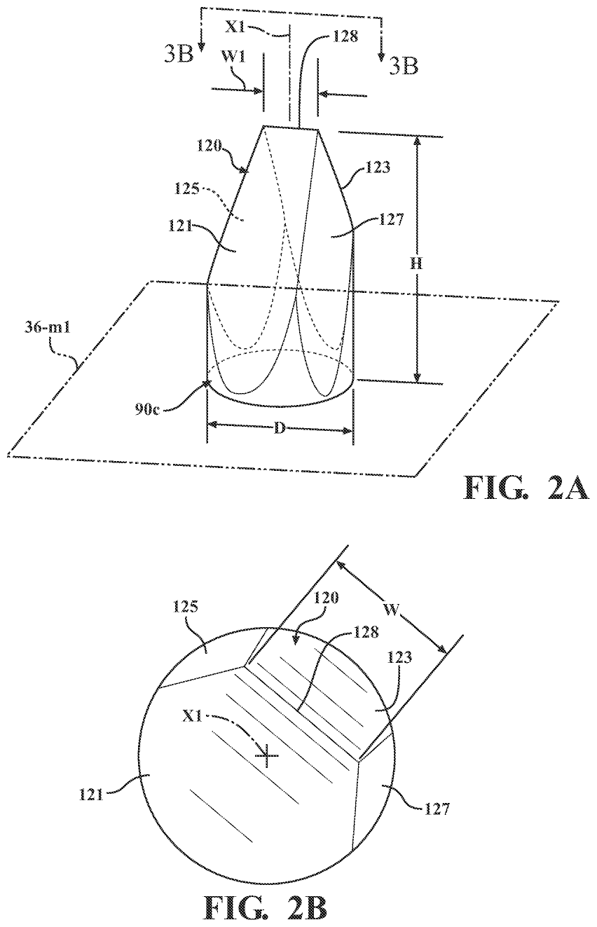

FIGS. 2A and 2B show one example of the microstructure of an individual unit cell 120. FIG. 2A is a schematic perspective view of an example unit cell in accordance with an embodiment described herein. FIG. 2B is a schematic plan view of the unit cell shown in FIG. 2A. In one or more arrangements, the unit cells of the plurality of unit cells described herein extend perpendicularly from the coating base portion 36m-1. The height H of a unit cell may be a distance that the unit cell extends above the base portion 36m-1, in a direction perpendicular to the coating base portion 36m-1. In particular arrangements, the height dimension H of each individual unit cell may be in the range 200 μmeter-800 μmeter inclusive. It has been found that variation of the unit cell heights within this range of dimensions provides the best overall conformity with ridges and irregularities in the skin surface of a finger pad or palm of an average user touching the microstructure.

In one or more arrangements, each of the unit cells 120 described herein may have a modified cylindrical configuration. In alternative arrangements, a unit cell may be formed by modifying another basic shape, such as an elongated rectangular prism, for example.

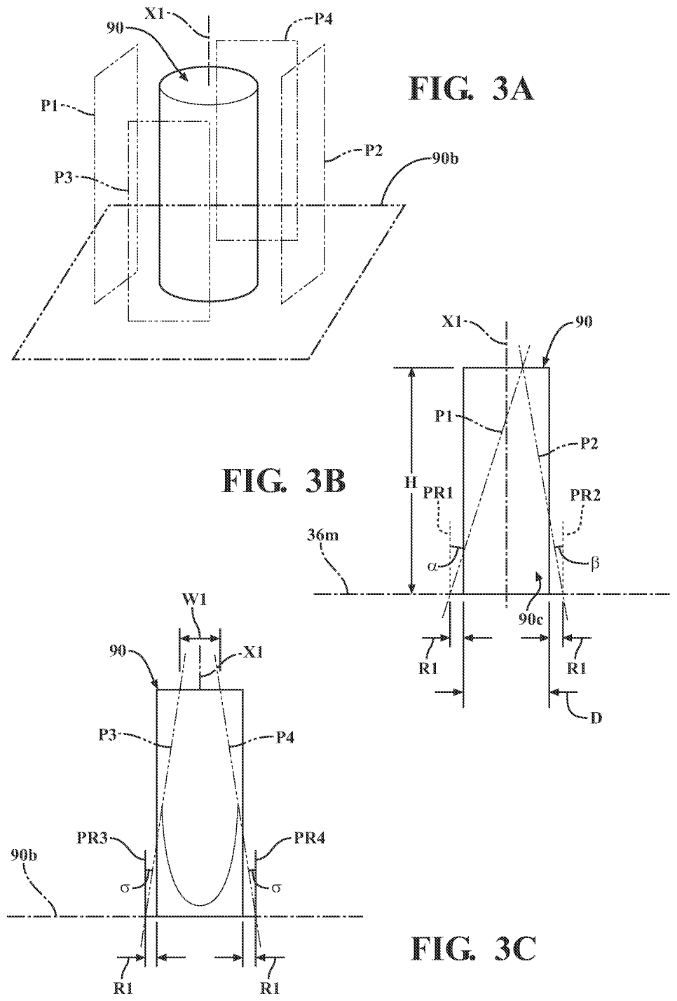

Referring to FIGS. 3A-3C, in modifying a cylindrical base shape, the geometry of an individual unit cell 120 may be formed by passing flat planes through a cylindrical member at various angles as described herein. For example, in one or more arrangements, the geometry may be formed in the following manner. Referring to FIGS. 3A-3C, a cylindrical member 90 may be provided having a diameter D and a central axis X1. In particular arrangements described herein, the diameter D may be in the range 200 μmeter-600 μmeter inclusive. A flat base plane 90b may extend perpendicular to the central axis X1 at an end of the cylindrical member 90 and parallel (or coplanar) with the outer surface of base portion 36m-1, thereby functionally representing the coating base portion. Four flat, mutually-orthogonal planes P1, P2, P3, and P4 may initially extend perpendicularly from the flat base plane 90b and may be positioned so as to enclose the cylindrical member 90. In one or more arrangements, each of the flat planes P1, P2, P3, and P4 may intersect the base plane 90b at a location spaced apart a radial distance R1 from an outer surface of the cylindrical member. In particular arrangements, the distance R1 may be in the range 0<R1<RC1, where RC1 is the radius of the cylindrical member 90.

Each of the flat planes P1, P2, P3, and P4 may then be rotated relative to a respective reference plane extending perpendicular to the base plane 90b, to an angle within a specified range, so as to intersect and cut through a portion of the cylindrical member 90. For example, referring to FIG. 3B, a first flat plane P1 may be rotated to intersect and cut the cylindrical member 90. The first plane P1 may be rotated to a first angle α with respect to a first reference plane PR1 extending parallel to the cylindrical member central axis X1 (or perpendicularly with respect to the base plane 90b), thereby defining a first flat surface 121 (FIG. 2A). In particular arrangements, the angle α may be in the range 30°-60° inclusive. It has been found that variation of the first flat surface orientations within this range provides the best overall conformity with ridges and irregularities in the skin surface of a finger pad or palm of an average user touching the microstructure.

Similarly, a second plane P2 (which originally extended parallel to the first plane P1) may be rotated to an angle β with respect to a second reference plane PR2 also extending parallel to the cylindrical member central axis X1 (or perpendicularly with respect to the base plane 90b), to intersect the cylindrical member 90 and the rotated first plane P1, thereby defining a second flat surface 123 (FIG. 2A) positioned opposite the first flat surface 121. In particular arrangements, the angle β may be in the range 10°-50° inclusive. It has been found that variation of the second flat surface orientations within this range provides the best overall conformity with ridges and irregularities in the skin surface of a finger pad or palm of an average user touching the microstructure.

Referring now to FIGS. 2A, 3A and 3C, in one or more particular arrangements, each unit cell 120 may further include a third flat surface 125 extending at a third angle σ with respect to a third reference plane PR3 extending perpendicularly with respect to the base plane 90b and perpendicularly with respect to the first reference plane PR1. In addition, each unit cell 120 may further include a fourth flat surface 127 positioned opposite the third flat surface 125 and also extending at the third angle σ with respect a fourth reference plane PR4 extending perpendicularly with respect to the base plane 90b and perpendicularly with respect to the first reference plane PR1. To form the third and fourth flat surfaces 125 and 127, the third flat plane P3 may be rotated to the third angle σ with respect to the third reference plane PR3, thereby defining the third flat surface 125. Also, the fourth flat plane P4 may be rotated to the angle σ with respect to the fourth reference plane PR4, thereby defining the fourth flat surface 127.

Referring to FIG. 2A, the intersection of the first, second, third, and fourth flat surfaces 121, 123, 125, 127 may form a contact edge 128 of the unit cell 120. The contact edge 128 may be structured to contact a portion of a user's skin surface, such as a ridge located on a pad of the user's fingertip. The contact edge may have a width W1. In one or more arrangements, the unit cells 120 of the microstructure 201 are formed from the same material. In one or more arrangements, the unit cells 120 of the microstructure 201 are formed from the same material as the coating base portion 36m-1.

Referring to FIG. 2A, each unit cell based on a cylindrical member as previously described may include a cylindrical bottom portion 90c operably connecting the unit cell with the coating base portion 36m-1. Each unit cell bottom portion 90c may have the diameter D.

In one or more arrangements described herein, dimensions of the unit cells 120 in an arrangement of unit cells forming an overall microstructure 201 may be varied to control the unit cell size characteristics (such as unit cell height H, bottom portion diameter D, and contact edge width W1, for example) to introduce controlled variability into the overall microstructure 201. At the same time, the overall shapes of the unit cells 120 may remain the same (i.e., the same features may appear in all of the unit cells even if common features differ in size from one cell to another). The controlled variability may enable the overall microstructure to accommodate a wide variety of different finger ridge sizes and spacings. Because finger skin ridge sizes and spacings vary between different users, the microstructures described herein may reduce contact resistance for a broad spectrum of different users.

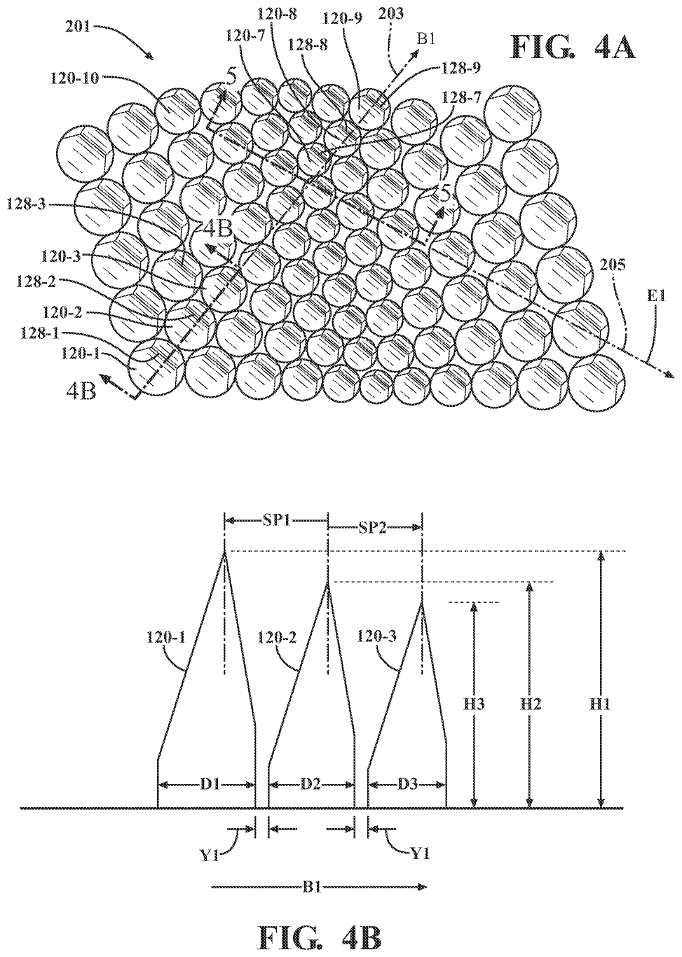

For example, FIG. 4A is a schematic plan view of a portion of an exemplary overall microstructure showing a possible size progression of the constituent unit cells, starting with unit cell 120-1 and proceeding in a direction B1. FIG. 4B is a schematic side cross-sectional view of a portion of the unit cells shown in FIG. 4A.

Referring to FIGS. 4A and 4B, unit cell height H, bottom portion diameter D, and the other dimensions of the unit cells may be reduced proportionately so that the overall sizes of the unit cells decrease proceeding along a plane 203 in direction B1. For example, FIGS. 4A and 4B show an arrangement wherein diameters D of successive unit cell bottom portions proceeding in direction B1 along the plane 203 extending perpendicular to the coating base portion 36m-1 decrease from a relatively greater diameter D1 to a relatively lesser diameter D3. Thus, starting with a first unit cell 120-1, the diameters D of successive cylindrical bottom portions may be reduced in a progression proceeding along the plane 203. For example, the diameter of the cell cylindrical bottom portion of unit cell 120-1 may be D1, the diameter of the cylindrical base portion of unit cell 120-2 may be D2, and the diameter of the cylindrical base portion of unit cell 120-3 may be D3, where D1>D2>D3. In addition, in particular examples, the succeeding diameters D1, D2, D3 may decrease by the same amount with each step. The gradual reduction in size of the unit cells along the plane 203 also enables a reduction in the distances between—the respective central axes of the bottom portions of successive pairs of unit cells.

In addition, in one or more arrangements, at least a portion of the unit cells 120 may be arranged so that diameters of successive unit cell bottom portions proceeding in a direction along a plane perpendicular to the coating base portion 36m-1 increase from a relatively lesser diameter to a relatively greater diameter. Thus, as seen in FIG. 4A, proceeding along plane 203 in direction B1, the diameter of the bottom portion of unit cell 120-8 may be greater than the diameter of the bottom portion of unit cell 120-7, and the diameter of the bottom portion of unit cell 120-9 may be greater than the diameter of the bottom portion of unit cell 120-8.

Thus, in the manner described above, the diameters D of the unit cell bottom portions may repeatedly progress from larger to smaller and vice versa over the extent of the overall microstructure.

Referring again to FIGS. 4A and 4B, in one or more arrangements, at least a portion of the unit cells 120 in the overall microstructure 201 may be arranged so that heights H of successive unit cells proceeding in a direction along a flat plane extending perpendicular to the coating base portion 36m-1 decrease from a relatively greater height to a relatively lesser height. Thus, for example, starting with a first unit cell 120-1, the heights H of successive unit cells may be reduced in a progression proceeding along the plane 203 in direction B1. Thus, the height of unit cell 120-1 may be H1, the height of the unit cell 120-2 may be H2, and the height of the unit cell 120-3 may be H3, where H1>H2>H3. In addition, in particular examples, the succeeding heights H1, H2, H3 may decrease by the same amount with each step.

Also, in one or more arrangements, at least a portion of the unit cells 120 may be arranged so that heights of successive unit cells proceeding in a direction along a flat plane extending perpendicular to the coating base portion 36m-1 increase from a relatively lesser height to a relatively greater height. Thus, for example, with unit cell 120-9 being farther along the plane 203 in direction B1, a height of unit cell 120-9 in FIG. 4A may be greater than the height of unit cell 120-8. Gradual variations in the unit cell heights are another way of introducing controlled variability into the contour formed by the local and overall microstructures.

In one or more arrangements, as the unit cells 120 become progressively smaller when proceeding in direction B1, the positions of the unit cells relative to each other may be adjusted to occupy the available space on the surface of the coating base portion as shown in FIG. 4A.

In one or more particular arrangements, unit cells of the plurality of unit cells may be arranged spaced apart from each other. Referring to FIG. 4B, for example, in one or more arrangements, the unit cells may be spaced apart equidistantly a distance of Y1 (i.e., an outer surface of each unit cell at its bottom portion may be spaced apart an equal distance from all adjacent unit cells, within applicable tolerance limits).

In one or more particular arrangements, the diameters D of the unit cell bottom portions of an arrangement of unit cells in an overall microstructure are equal (within applicable manufacturing tolerances) in all directions.

In one or more particular arrangements, the heights H of the unit cells of the plurality of unit cells are equal (within applicable manufacturing tolerances).

Referring to FIG. 4A, in one or more particular arrangements, the unit cells 120 of the in an overall microstructure 201 are arranged so that their contact respective edges extend parallel (or substantially parallel) to each other (within applicable tolerance limits). In particular arrangements, at least a portion of the unit cells 120 may be arranged so that a spacing between contact edges of successive unit cells proceeding in a direction along the a plane perpendicular to the coating base portion decreases from a relatively greater spacing to a relatively lesser spacing. For example, referring again to FIGS. 4A and 4B, proceeding along plane 203 in direction B1, a spacing between contact edges 128-1 and 128-2 may be SP1, while a spacing between contact edges 128-2 and 128-3 may be SP2, where SP1>SP2. In addition, in particular arrangements, at least a portion of the unit cells 120 are arranged so that spacings between contact edges of successive unit cells proceeding in a direction along a plane perpendicular to the coating base portion increases from a relatively lesser spacing to a relatively greater spacing. For example, proceeding farther along plane 203 in direction B1, a spacing between contact edges 128-8 and 128-9 may be greater than a spacing between contact edges 128-7 and 128-8. Other arrangements are also possible.

Gradual variations in the unit cell contact edge spacings is yet another way of introducing controlled variability into the contours formed by the local and overall microstructures. The spacing between contact edges of any two adjacent unit cells may be taken as a perpendicular distance between planes extending along the respective contact edges and perpendicular to the coating base portion 36m-1. In particular arrangements, the spacing between contact edges of adjacent unit cells is in the range 200 μ-meter to 800 μ-meter. It has been found that variation of the contact edge spacing within this range of dimensions provides the best overall conformity with ridges and irregularities in the skin surface of a finger pad or palm of an average user touching the microstructure.

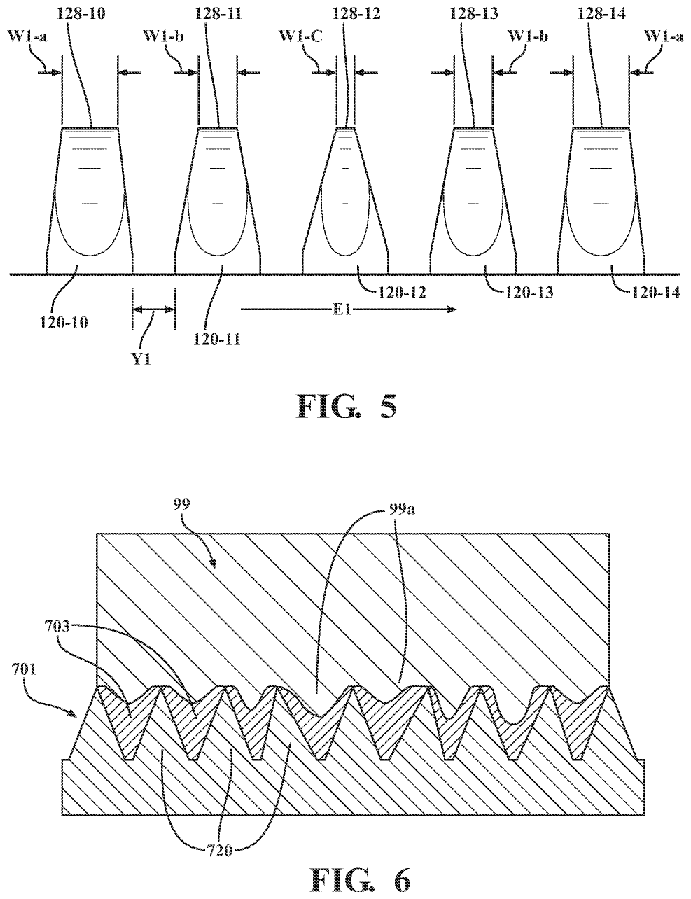

In particular arrangements, in another method of introducing a controlled variability into the contours formed by the local and overall microstructures, a grouping of adjacent unit cells may have the same height H. In addition, each unit cell contact edge has an associated width W1, and at least a portion of the unit cells are arranged so that the contact edge widths of successive unit cells proceeding in a first direction along a plane perpendicular to the coating base portion decrease from a relatively greater width to a relatively lesser width. For example, FIG. 5 is a schematic front view of a plurality of unit cells of an overall microstructure in accordance with an embodiment described herein, showing progressions in widths of the unit cell contact edges. Referring to FIG. 5, proceeding along the plane 205 in direction E1, the width W1-a of contact edge 128-10 of unit cell 120-10 may be greater than the width W1-b of contact edge 128-11 of adjacent unit cell 120-11. Similarly, the width W1-b of contact edge 128-11 of unit cell 120-11 may be greater than the width W1-c of contact edge 128-12 of adjacent unit cell 120-12.

In addition, in particular arrangements, at least a portion of the unit cells are arranged so that contact edge widths of successive unit cells proceeding in a direction along a plane perpendicular to the coating base portion increase from a relatively lesser width to a relatively greater width. For example, referring to FIG. 5, proceeding farther in direction E1, the width W1-b of contact edge 128-13 of unit cell 120-13 may be greater than the width W1-c of contact edge 128-12 of adjacent unit cell 120-12. Similarly, the width W1-a of contact edge 128-14 of unit cell 120-14 may be greater than the width W1-b of contact edge 128-13 of adjacent unit cell 120-13. As used herein, the term “adjacent” is understood to mean “immediately preceding or following”.

Gradual variations in the widths of the contact edges of the unit cells is yet another way of introducing controlled variability into the contours formed by the local and overall microstructures.

Referring now to FIG. 6, in an alternative embodiment 701 of an overall microstructure, the unit cells 720 may be formed from the same material. In addition, the material forming the unit cells 720 may have a first hardness. Also, all gaps between adjacent unit cells may be filled in with a conductive material 703 having a second hardness less than the first hardness. For example, the unit cell material may have a different Shore hardness value than the gap-filling material 703. Both the unit cell material and the gap-filler material may incorporate conductive fillers to minimize contact resistance. The relatively softer material 703 may resiliently deform responsive to contact with the skin ridges 99a of a user's finger 99, thereby increasing the contact area (and decreasing the contact resistance) between the user's skin and the coating.

In additional aspects, an embodiment of the electrode assembly described herein may be incorporated onto an electrodermal activity sensor.

In the above detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The terms “a” and “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e. open language). The phrase “at least one of . . . and . . . ” as used herein refers to and encompasses any and all possible combinations of one or more of the associated listed items. As an example, the phrase “at least one of A, B and C” includes A only, B only, C only, or any combination thereof (e.g. AB, AC, BC or ABC).

Aspects herein can be embodied in other forms without departing from the spirit or essential attributes thereof. Accordingly, reference should be made to the following claims, rather than to the foregoing specification, as indicating the scope of the invention.

What is claimed is:

1. An electrode assembly structured for use in detecting electrodermal activity (EDA), the assembly comprising:
a substrate;
at least two separate electrodes affixed to the substrate; and
a separate electrically-conductive coating covering each electrode, each coating including:
a base portion; and
a plurality of unit cells extending from the base portion.

2. The electrode assembly of claim 1, wherein the unit cells of the plurality of unit cells are spaced apart equidistantly.

3. The electrode assembly of claim 1, wherein the unit cells of the plurality of unit cells extend perpendicularly from the coating base portion.

4. The electrode assembly of claim 1, wherein the coating base portion has a uniform thickness.

5. The electrode assembly of claim 1, wherein the plurality of unit cells, in combination, form an overall microstructure supported by the coating base portion.

6. The electrode assembly of claim 1, wherein each unit cell of the plurality of unit cells comprises:
a first flat surface extending at a first angle with respect to a first reference plane extending perpendicularly with respect to the coating base portion; and
a second flat surface positioned opposite the first flat surface and extending at a second angle with respect to a second reference plane extending perpendicularly with respect to the coating base portion.

7. The electrode assembly of claim 6, wherein each unit cell of the plurality of unit cells further comprises:
a third flat surface extending at a third angle with respect to a third reference plane extending perpendicularly with respect to the coating base portion and orthogonally with respect to the first reference plane; and
a fourth flat surface positioned opposite the third flat surface and extending at the third angle with respect a fourth reference plane extending perpendicularly with respect to the coating base portion and orthogonally with respect to the first reference plane.

8. The electrode assembly of claim 7, wherein each unit cell includes a contact edge, each contact edge having an associated width, and wherein at least a portion of the unit cells are arranged so that contact edge widths of successive unit cells proceeding in a direction along a plane perpendicular to the coating base portion decrease from a relatively greater width to a relatively lesser width.

9. The electrode assembly of claim 7, wherein each unit cell includes a contact edge, each contact edge having an associated width, and wherein at least a portion of the unit cells are arranged so that contact edge widths of successive unit cells proceeding in a direction along a plane perpendicular to the coating base portion increase from a relatively lesser width to a relatively greater width.

10. The electrode assembly of claim 1, wherein each unit cell of the plurality of unit cells has an associated height, and wherein at least a portion of the unit cells are arranged so that heights of successive unit cells proceeding in a direction along a flat plane extending perpendicular to the coating base portion decrease from a relatively greater height to a relatively lesser height.

11. The electrode assembly of claim 1, wherein each unit cell of the plurality of unit cells has an associated height, and wherein at least a portion of the unit cells are arranged so that heights of successive unit cells proceeding in a direction along a flat plane extending perpendicular to the coating base portion increase from a relatively lesser height to a relatively greater height.

12. The electrode assembly of claim 1, wherein each unit cell of the plurality of unit cells has an associated height, and wherein heights of the unit cells of the plurality of unit cells are equal.

13. The electrode assembly of claim 1, wherein each unit cell of the plurality of unit cells comprises a cylindrical bottom portion operably connecting the unit cell with the coating base portion, each unit cell bottom portion having an associated equal diameter.

14. The electrode assembly of claim 1, wherein each unit cell of the plurality of unit cells comprises a cylindrical bottom portion operably connecting the unit cell with the coating base portion, wherein each unit cell bottom portion has an associated diameter, and wherein at least a portion of the unit cells are arranged so that diameters of successive unit cell bottom portions proceeding in a direction along a plane perpendicular to the coating base portion decrease from a relatively greater diameter to a relatively lesser diameter.

15. The electrode assembly of claim 1, wherein each unit cell of the plurality of unit cells comprises a cylindrical bottom portion operably connecting the unit cell with the coating base portion, wherein each unit cell bottom portion has an associated diameter, and wherein at least a portion of the unit cells are arranged so that diameters of successive unit cell bottom portions proceeding in a direction along a plane perpendicular to the coating base portion increase from a relatively lesser diameter to a relatively greater diameter.

16. The electrode assembly of claim 1, wherein the unit cells of the plurality of unit cells are formed from a same material, wherein the material forming the unit cells has a first hardness, wherein each unit cell of the plurality of unit cells is spaced apart at least a predetermined distance from any adjacent unit cell so as to provide a gap therebetween, and wherein all gaps between adjacent unit cells are filled in with a conductive material having a second hardness less than the first hardness.

17. An electrodermal activity sensor including an electrode assembly in accordance with claim 1.

18. An electrode assembly structured for use in detecting electrodermal activity (EDA), the assembly comprising:
a substrate;
at least two separate electrodes affixed to the substrate;
a separate electrically conductive coating covering each electrode, each coating including:
a base portion; and
a plurality of unit cells extending from the base portion,
all of the unit cells being formed from a same material, the material forming the unit cells having a first hardness, wherein each unit cell is spaced apart at least a predetermined distance from any adjacent unit cell so as to provide a gap therebetween, and wherein all gaps between adjacent unit cells are filled in with a conductive material having a second hardness less than the first hardness.
